(19) United States
(12) Patent Application Publication
(10) Pub. No.: US 2009/0024157 A1
(43) Pub. Date: Jan. 22, 2009

(54) EMBOLIC PROTECTION DEVICE WITH OPEN CELL DESIGN

(75) Inventor: BORIS ANUKHIN, SAN JOSE, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: ABBOTT LABORATORIES, ABBOTT PARK, IL (US)

(21) Appl. No.: 11/779,808

(22) Filed: Jul. 18, 2007

(51) Int. Cl. A61F 2/01; A61B 19/00

(52) U.S. Cl. 606/200; 128/898

(57) ABSTRACT

A cage and sleeve assembly for an embolic filtering device used to filter embolic particles from a body vessel has a strut assembly that is movable between an unexpanded position and an expanded position. The struts are configured to form a cage having an open cell design. The open cell design provides a filter cage with increased radial flexibility that also increases the contact with a vessel wall while reducing the landing zone length. Such an open cell design may have one or more rings that are not connected together at each vertex, and may be constructed from one ring. The sleeve assembly of the embolic filtering device may be joined to the filter cage at each vertex of the open cell or along the periphery of the open cell.

### BACKGROUND OF THE INVENTION

The present invention relates generally to filtering devices used when an interventional procedure is being performed in a stenosed or occluded region of a body vessel. The filtering devices capture embolic material that may be created and released into the vessel during the procedure. The present invention is more particularly directed to an embolic filtering device made with a flexible, bendable and expandable cage or basket, configured with an open cell design. The concepts presented herein may be applied to any of a wide variety of other devices.

Numerous approaches have been developed for treating occluded blood vessels, usually involving the percutaneous introduction of an interventional device into the lumen of the artery, usually by a catheter. One widely known and medically accepted procedure is balloon angioplasty, in which an inflatable balloon is introduced within the stenosed region of the blood vessel to dilate the occluded vessel. The balloon dilatation catheter is initially inserted into the patient's arterial system and is advanced and manipulated into the area of stenosis in the artery. The balloon is inflated to compress the plaque and to press the vessel wall radially outward to increase the diameter of the blood vessel, resulting in increased blood flow. The balloon is then deflated to a small profile so that the physician can withdraw the dilatation catheter from the patient's vasculature. As should be appreciated by those skilled in the art, while the above-described procedure is typical, it is not the only method used in angioplasty.

Another procedure is laser angioplasty, which utilizes a laser to ablate the stenosis by super heating and vaporizing the deposited plaque. Still another method is atherectomy, in which cutting blades are rotated to shave the deposited plaque from the arterial wall. A catheter is usually used to capture the shaved plaque or thrombus from the bloodstream.

In the procedures of this kind, abrupt reclosure of the artery may occur, or restenosis of the artery may develop over time, requiring another angioplasty procedure, a surgical bypass operation, or some other method of repairing or strengthening the area. To reduce the likelihood of abrupt reclosure and to strengthen the area, a physician can implant an intravascular prosthesis for maintaining vascular patency, commonly known as a stent, inside the artery across the lesion. The stent can be crimped tightly onto the balloon portion of the catheter and transported in its delivery diameter through the patient's vasculature. At the deployment site, the stent is expanded to a larger diameter, often by inflating the balloon portion of the catheter. Alternatively, the stent may be of the self-expanding type, such that a balloon to expand the stent is not needed.

The above non-surgical interventional procedures, when successful, avoid the necessity of major surgical operations. However, there is one common problem associated with all of these non-surgical procedures, namely, the potential release of embolic debris into the bloodstream that can occlude distal vasculature and cause significant health problems to the patient. For example, pieces of plaque material are sometimes generated during a balloon angioplasty procedure and become released into the bloodstream. Or, during deployment of a stent, the metal struts of the stent may cut into the stenosis and shear off pieces of plaque that can travel downstream and lodge somewhere in the patient's vascular system. Also, while complete vaporization of plaque is the intended goal during laser angioplasty, sometimes particles are not fully vaporized and enter the bloodstream. Likewise, not all of the emboli created during an atherectomy procedure may be drawn into the catheter and, as a result, enter the bloodstream as well.

When any of the above-described procedures are performed in the carotid arteries, the release of emboli into the circulatory system can be extremely dangerous and sometimes fatal to the patient. Debris carried by the bloodstream to distal vessels of the brain can cause cerebral vessels to occlude, resulting in a stroke, and in some cases, death. Therefore, although cerebral percutaneous transluminal angioplasty has been performed in the past, the number of procedures performed has been somewhat limited due to the justifiable concern over an embolic stroke occurring should embolic debris enter the bloodstream and block vital downstream blood passages.

Medical devices have been developed to deal with the problem of debris or fragments entering the circulatory system following vessel treatment utilizing any one of the above-identified procedures. One approach that has been attempted is to cut any debris into minute sizes, which pose little chance of becoming occluded in major vessels within the patient's vasculature. However, it is often difficult to control the size of the fragments that are formed, and the potential risk of vessel occlusion still exists, making such a procedure in the carotid arteries a high-risk proposition.

Other techniques include the use of catheters with a vacuum source that provides temporary suction to remove embolic debris from the bloodstream. There can be complications associated with such systems, however, if the vacuum catheter does not remove all of the embolic material from the bloodstream. Also, a powerful suction could cause trauma to the patient's vasculature.

Another technique that has had some success utilizes a filter or trap downstream or distal from the treatment site to capture embolic debris in the bloodstream. The placement of a filter in the patient's vasculature during treatment of the vascular lesion can reduce the presence of the embolic debris in the bloodstream. Such embolic filters are usually delivered in a collapsed position through the patient's vasculature and then expanded to trap the embolic debris. Some of these embolic filters are self expanding and utilize a restraining sheath that maintains the expandable filter in a collapsed position until it is ready to be expanded within the patient's vasculature. The physician can retract the proximal end of the restraining sheath to expose the expandable filter, causing the filter to expand at the desired location. Once the procedure is completed, the filter can be collapsed, and the filter (with the trapped embolic debris) can then be removed from the vessel.

Turning now to the structure of the embolic filter, such filters include an expandable filter cage assembly for retaining the embolic debris. The distal ends (furthest into the body) and proximal ends (closest to outside of the body) of the expandable filter cage include struts that may attach to respective distal and proximal sleeves. The geometry of the filter cage is constructed from “cells,” which are formed by two sets of rings connected together. Rings are formed from the struts, which are the smallest entity of the filter cage. Heretofore, such filter cages have been configured with a “closed cell design.”

A closed cell design (FIGS. 10 and 12) is a configuration of the filter cage assembly with struts constructed to form a closed loop in the shape of (but not limited to) a diamond, rhombus, kite, parallelogram or combinations thereof. A closed cell filter cage design can not be constructed from one ring. Closed cell designs are constructed from two sets of rings connected at every vertex. In addition, a closed cell design must have at least two rings, forming looped geometry. Although a closed cell design may provide an excellent radial force, a closed cell design does not have enough flexibility so as to have a poor vasculature wall opposition due to the natural geometric properties of the closed cell. In addition, a closed cell configuration has a significant “landing zone length,” which also decreases flexibility and reduces vasculature wall opposition.

What has been needed and heretofore unavailable is an embolic filter having an improved filter cage that overcomes the deficiencies of a closed cell configuration so as to increase flexibility and improve vasculature wall opposition of the filter cage. The present invention disclosed herein satisfies these and other needs.

### SUMMARY OF THE INVENTION

The present invention provides an improved filter cage of an embolic filter assembly that overcomes the deficiencies of a closed cell configuration so as to increase flexibility and improve vasculature wall opposition of the filter cage. The improvement of the present invention is directed to the configuration of the filter cage by using an open cell design.

Embolic filters of the present invention may be used to treat a variety of conditions with the vasculature of a patient. For example, carotid arteries on both sides of the neck serve as the main conduits for blood flow to the brain. Fatty deposits of cholesterol, calcium and blood platelets (atherosclerotic plaque) can build up inside them, reducing flow and causing Transient Ischemic Attacks (TIA) or an ischemic stroke, the most common type of stroke. Stenting is well known solution to treat this problem. Unfortunately, during the procedure little pieces of the deposits will break away and flow directly into the brain causing the complications. To reduce the amount of emboli flow into the brain, a filter may be being temporary deployed above the stented region capturing most or all of the debris. Often during the procedure, the filter is not being fully expanded, thereby allowing the emboli to pass between the filter and the artery wall. One of the solutions to this problem recognized by the present invention is to change the design of the filter cage from a closed cell design to an open cell design. An open cell design will allow the cage to be more flexible radially, better contact the wall and also minimize the landing zone length.

In one embodiment of the present invention, a cage and sleeve assembly for an embolic filtering device used to filter embolic particles from a body vessel includes a strut assembly that is movable between an unexpanded position and an expanded position. The assembly includes struts that form a cage, with the struts. The struts of the filter cage are at least partially made of a medical grade metal or alloy, such as, but not limited to, nitinol (Ni—Ti), stainless steel, cobalt-chromium and titanium. One or more welds secures the strut ends in the sleeve assembly. The embolic filter cage may be formed by laser cutting a hypotube of a selected metal or alloy into a desired configuration of the filter cage.

Generally, an open cell filter cage design of the present invention is the design is configured with struts constructed not to form a closed loop in the shape of (but not limited) the diamond, rhombus, kite, parallelogram etc. or their combinations. Such an open cell design may have at least one or more rings that preferably are not connected together at each vertex. An open cell filter cage design, however, can be constructed from one ring. Such a embolic filter cage will reduce the landing zone of the filter cage and will improve the contact of the filter cage to the vasculature wall.

The embolic filtering device of the present invention is configured from a filter cage assembly that is movable between an unexpanded position and an expanded position. The filter cage includes an open cell central ring, a plurality of proximal struts connected to the central ring and a plurality of distal struts connected to the central ring, and a sleeve assembly formed around the distal struts of the filter cage assembly. The open cell central ring may be formed with six proximal vertexes each joined to a proximal strut and six distal vertexes each joined to a distal strut such that the sleeve assembly is secured to each of the distal vertexes or along the periphery of the open cell. The sleeve assembly may be configured with a plurality of perforations. The filter cage and open cell may be formed from a nickel-titanium alloy or other self-expanding material, or may be formed from stainless steel, a cobalt-chromium alloy or other suitable biocompatible material.

The present invention further includes a filtering system including a filter cage assembly, a sleeve assembly, a guidewire disposed within the filter cage and the sleeve assembly and a sheath. The sheath retains the filter cage, which is movable between an unexpanded position and an expanded position and includes an open cell central ring. The filter cage further includes a plurality of proximal struts each having a proximal portion and a distal portion such that each distal portion is connected to one of a plurality of proximal vertexes formed in the central ring, and a plurality of distal struts each having a proximal portion and a distal portion such that each proximal portion is connected to one of a plurality of distal vertexes formed in the central ring. The sleeve assembly is formed around the distal struts of the filter cage assembly and secured to the open cell ring. The filtering system of the present invention may further include an obturator configured to enclose the distal portions of the distal struts, and may include a cover configured to enclose the proximal portions of the proximal struts.

A method of the present invention includes providing a filter cage assembly that is movable between an unexpanded position and an expanded position, wherein the filter cage includes an open cell central ring, a plurality of proximal struts each having a proximal portion and a distal portion such that each distal portion is connected to one of a plurality of proximal vertexes formed in the central ring, and a plurality of distal struts each having a proximal portion and a distal portion such that each proximal portion is connected to one of a plurality of distal vertexes formed in the central ring. The method further includes providing a sleeve assembly formed around the distal struts of the filter cage assembly and secured to the open cell ring, providing a guidewire having a proximal portion and a distal portion disposed within the filter cage and the sleeve assembly and providing a sheath configured to contain the filter cage and sleeve assembly when the filter cage is in the unexpanded position. In the method of the present invention, the guidewire is inserted into the vasculature of a patient and the proximal portion of the guidewire is manipulated such that the distal portion of the guidewire is positioned at a desired location in the vasculature. The sheath containing the distal portion of the filter cage and sleeve assembly is then inserted over the guidewire so as to position the distal portion of the sheath at the desired location in the vasculature and the sheath is moved in a proximal direction so as to allow the filter cage to form in its expanded position.

It is to be understood that the present invention is not limited by the configurations of the filter cage described herein or by the materials of construction disclosed. The present invention can be used in arteries, veins, and other body vessels. The inventive concept may also be extended beyond embolic filtering devices, and may encompass other medical and non-medical devices. Other features and advantages of the present invention will become more apparent from the following detailed description of the invention, when taken in conjunction with the accompanying exemplary drawings.

### BRIEF DESCRIPTION OF THE DRAWINGS

FIG. 1 is a perspective view of an embolic filtering device having an open cell filter cage assembly according to the present invention, wherein the sleeve assembly is joined at each apex of the filter cage open cell.

FIG. 2 is a perspective view of the embolic filtering device of FIG. 1 in its expanded configuration with the filter sleeve removed to better show the open cell filter cage assembly.

FIG. 3 is a perspective view of an embolic filtering device having an open cell filter cage assembly according to the present invention, wherein the sleeve assembly is joined along the periphery of the filter cage open cell.

FIG. 4 is a side plan view, partially in cross section, of an embolic filtering device including a restraining sheath of the present invention as it is being delivered within a vasculature and downstream from an area to be treated.

FIG. 5 depicts a side plan view of an embolic filtering device of the present invention as the open cell filter cage assembly expands within the vasculature.

FIG. 6 is a side plan view of an embolic filtering device of the present invention deployed in its expanded position within a vasculature and depicting the filtering process.

FIG. 7 is a perspective view of an open cell filter cage assembly according to the present invention.

FIG. 8 is a side plan view depicting the proximal strut ends of a open cell filter cage assembly of an embolic filtering device of the present invention.

FIG. 9 is a cross-sectional view of one embodiment of the distal portion of an embolic filtering device of the present invention.

FIG. 10 is a side plan view of a closed cell filter cage assembly that is known in the art.

FIG. 11 is a side plan view of an open cell filter cage assembly according to the present invention.

FIG. 12 is a schematic representation of closed cell configurations that are known in the art.

FIG. 13 is a schematic representation of open cell configurations according to the present invention.

FIG. 14 is a schematic representation of closed cell configurations juxtaposed against open cell configurations.

FIG. 15 is a perspective view of one embodiment of an open cell filter cage assembly according to the present invention depicted as cut from a hypotube and shown in a flattened representation.

### DETAILED DESCRIPTION OF THE PREFERRED EMBODIMENTS

The present invention is directed to an embolic filtering device having an open cell configuration. The embolic filtering device includes a filter cage and a sleeve assembly used to filter embolic particles from a body vessel. The filter cage includes a strut assembly that is movable between an unexpanded position and an expanded position. The strut assembly includes struts that form the filter cage having an open cell design. The open cell filter cage design of the present invention is configured with struts constructed not to form a closed loop or cell. Such an open cell design may have at least one or more rings that preferably are not connected together at each vertex. An open cell filter cage design, however, can be constructed from one ring. Such an embolic filter cage reduces the landing zone of the filter cage and improves the contact of the filter cage to the vasculature wall.

It should be appreciated that the embodiments of the embolic filtering device 20 described herein are illustrated and described by way of example only and not by way of limitation. Also, while the present invention is described in detail as applied to an artery of the patient, those skilled in the art will appreciate that it can also be used in other body vessels, such as the coronary arteries, carotid arteries, renal arteries, saphenous vein grafts and other peripheral arteries, as well as veins, the pulmonary system and other channels for bodily fluid or gas. Additionally, the present invention can be utilized when a physician performs any one of a number of interventional procedures, such as balloon angioplasty or atherectomy that generally require an embolic filtering device to capture embolic debris created during the procedure.

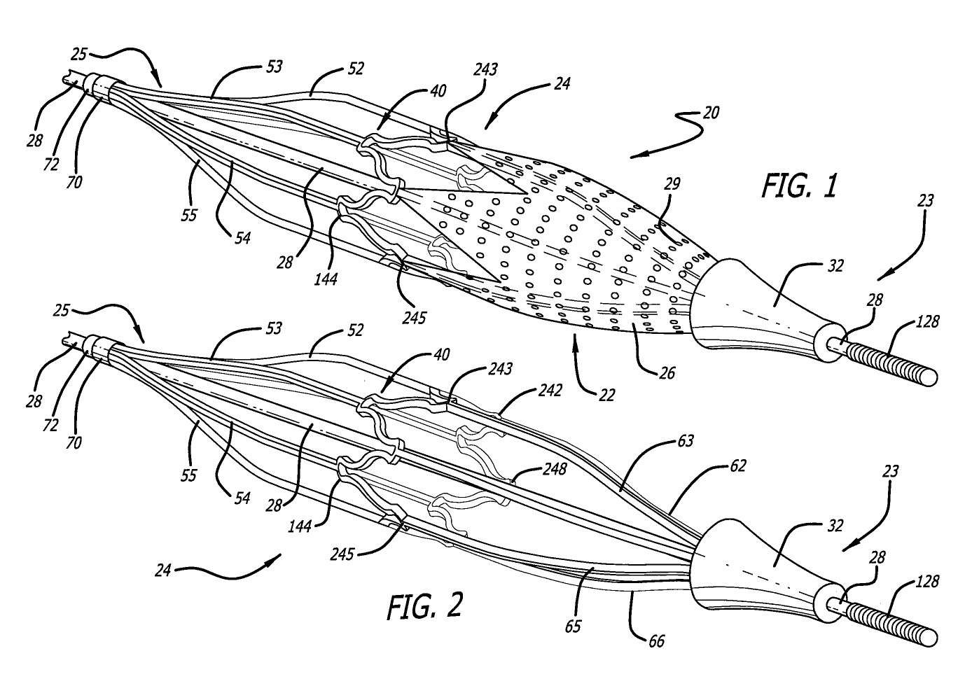

Turning now to the drawings, in which like reference numerals represent like or corresponding elements in the drawings, FIGS. 1-3 illustrate particular embodiments of an embolic filtering device 20 of the present invention. Such an embolic filtering device is designed to capture embolic debris that may be created and released into a body vessel during an interventional procedure. The embolic filtering device includes an expandable filter assembly 22 having a self-expanding or balloon-expandable basket or filter cage 24 and a sleeve assembly 26 attached thereto. In these particular embodiments, the filter assembly is rotatably mounted on the distal end of an elongated (solid or hollow) cylindrical tubular shaft, such as a guidewire 28 having a flexible tip 128.

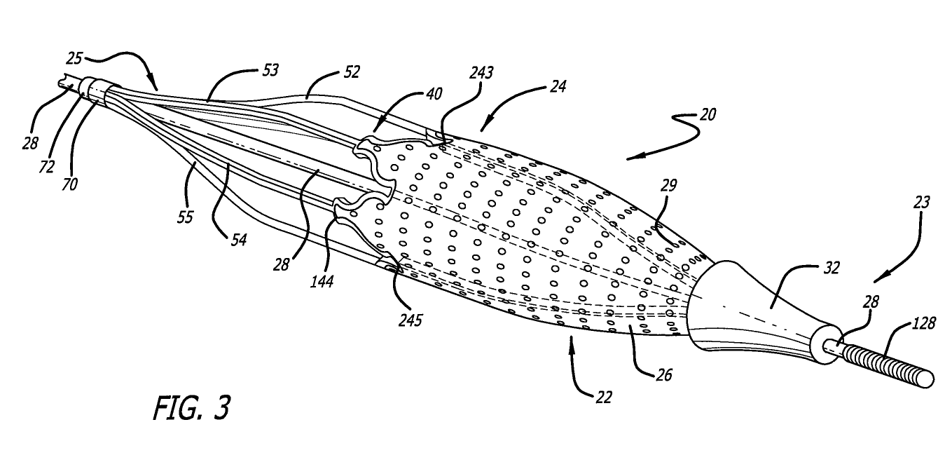

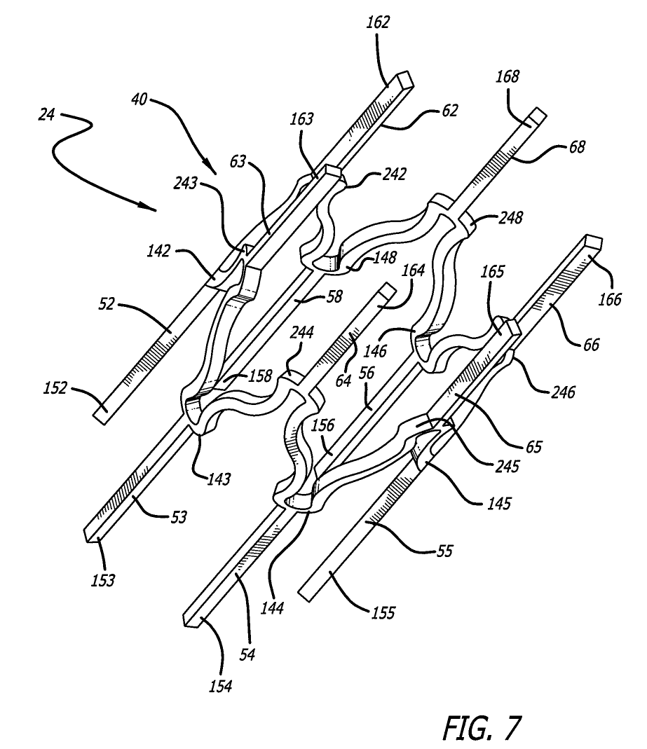

The filter cage 24 includes an open cell 40 having a plurality of proximal struts 52-58 and a plurality of distal struts 62-86 that, upon expansion of the open cell and struts, expand the filter sleeve 26 into its deployed position within the artery. Embolic particles created during the interventional procedure and released into the bloodstream are captured within the deployed filter sleeve. The filter sleeve may include perfusion openings 29, or other suitable perfusion means, for allowing blood flow through the filter assembly 22. As shown in FIG. 1, the filter sleeve may be joined at each apex 144, 243, 245 (see FIG. 7) of the open cell of the filter cage. Alternatively, and as shown in FIG. 3, the filter sleeve may be joined to essentially the entire circumference or periphery of the open cell of the filter cage.

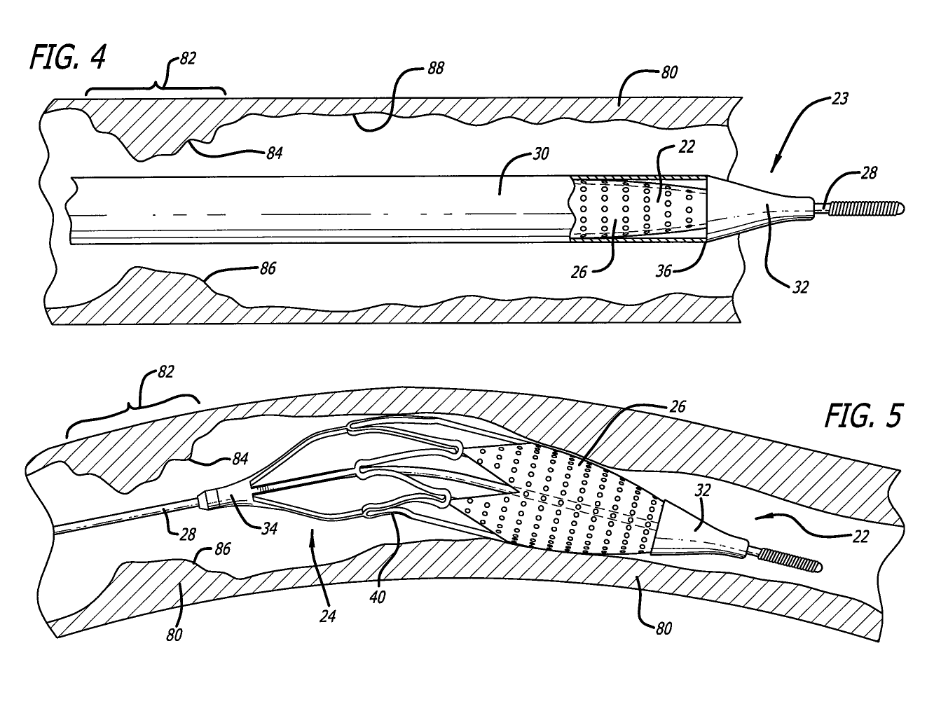

Referring also to FIG. 4, the guidewire 28 of the embolic filtering device 20 has a proximal end (not shown) that extends outside the patient and is manipulated by the physician to deliver the filter assembly 22 to the target lesion to be treated. A restraining or delivery sheath 30 extends coaxially along the guidewire in order to maintain an expandable filter assembly in its collapsed position until it is ready to be deployed within the patient's vasculature 80. A self-expanding filter assembly is deployed by the physician by retracting the restraining sheath 30 proximally to expose the expandable filter assembly. As the restraining sheath is retracted, the self-expanding filter cage 24 immediately begins to expand within the body vessel, causing the sleeve assembly 26 also to expand. As will be appreciated by those having ordinary skill in the art of medical devices, the embolic filtering device and filter assembly may be configured with a balloon catheter and inflation mechanism to expand a filter cage not configured from a self-expanding material.

A conical, frustum tapered, or other suitably shaped obturator 32 may be affixed to the distal portion 27 of the filter assembly 22 to prevent or reduce “snowplowing” of the embolic filtering device 20 into the vessel walls as it is being delivered through the vasculature 80. The obturator may also be configured to retain the distal ends of the distal struts 62-68 of the filter cage 24. In addition, the proximal portion 25 of the filter assembly may include a proximal cover 34 for protecting the proximal ends of the filter cage proximal struts 52-58. The obturator and proximal cover can be made from a soft polymeric material, such as Pebax 40D, and preferably have smooth surfaces to help the embolic filtering device travel through the vasculature and cross lesions. The obturator also helps to prevent the distal end 36 of the restraining sheath 30 from “digging” or “snowplowing” into the wall of the body vessel. The obturator may be fixedly or rotatably attached to the guidewire 28.

A collar 70 may be positioned on the guidewire 28 proximal of the proximal portion 25 of the filter assembly 22 to maintain the filter cage 24 rotatably fixed to the guidewire. A stop fitting 72 may be fixed to the guidewire proximal of the collar so as to allow the filter cage to rotate or “spin” freely on the guidewire, while restricting the longitudinal movement of the cage on the guidewire. This particular mechanism is just one way in which the filter cage can be mounted to the guidewire. Alternatively, the filter cage can be fixedly attached directly onto the guidewire so as not to permit the cage to rotate independently.

As shown in FIG. 5, the embolic filtering device 20 is configured to be delivered within an artery 80 or other body vessel of the patient. The filter assembly 22 is configured to expand within the patient's artery proximate to an area of treatment 82 in which atherosclerotic plaque 84, 86 has built up against the inside wall 88 of the vessel. The filter assembly is to be placed distal to, and downstream from, the area of treatment. For example, the therapeutic interventional procedure may include the implantation of a stent (not shown) to increase the diameter of an occluded artery and increase the flow of blood through the vessel.

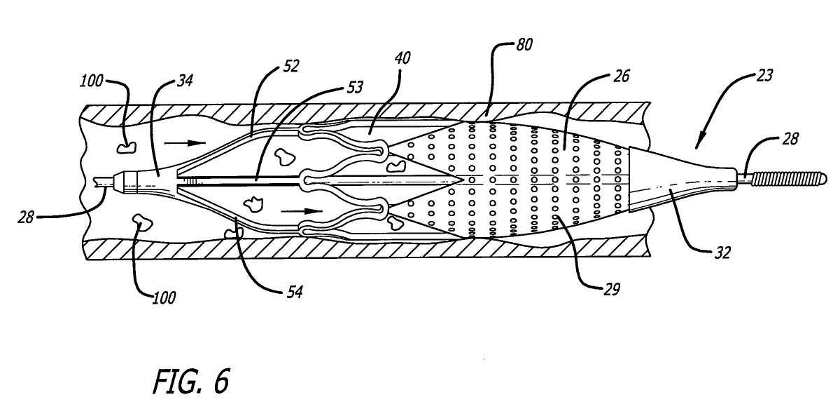

Referring to FIG. 6. the sleeve assembly 26 of the filter assembly 22 is configured to trap or catch embolic particles 100 that are larger than the perfusion openings 29, while allowing some blood to perfuse downstream to vital organs. Although not shown, after the deployment of the filter assembly, a balloon angioplasty catheter can be introduced within the patient's vasculature in a conventional Seldinger technique through a guiding catheter (not shown). The guidewire 28 is disposed through the area of treatment 82 and a dilatation catheter can be advanced over the guidewire 28 within the artery 80 until a balloon portion of the dilatation catheter is directly in the area of treatment. The balloon of the dilatation catheter can be expanded, expanding the plaque or other lesion 84, 86 against the wall 88 of the artery to expand the artery and reduce the blockage in the vessel at the area of treatment.

After the dilatation catheter is removed from the patient's vasculature, a stent (not shown) may be implanted in the area of treatment using over-the-wire or rapid exchange techniques to help hold open and maintain patency of this portion of the vessel and to help prevent restenosis from occurring in the area of treatment. The stent could be delivered to the area of treatment on a stent delivery catheter (not shown) that is advanced from the proximal end of the guidewire to the area of treatment. Any embolic debris created during the interventional procedure will be released into the bloodstream and should be captured by the filter 26. Once the procedure is completed, the interventional device may be removed from the guidewire. The filter assembly 22 can also be collapsed and removed from the artery 34, taking with it any embolic debris trapped within the filter element 26. A recovery sheath (not shown) can be delivered over the guidewire 28 to collapse the filter assembly 22 for removal from the patient's vasculature.

Referring now to FIG. 7 for exemplary purposes only, the present invention includes a filter cage assembly 24 that has been cut from a thin-walled tube of substrate material, (such as stainless steel, nitinol, cobalt-chromium, titanium and other metals or alloys) configured using a laser cutter or according to other methods known in the art. The filter cage includes a central open cell 40 with proximal struts and distal struts. The number of struts formed on the filter cage can be any number (including, but not limited to, 4, 6, 8 or 10) that will provide sufficient expandability within the artery or other vessel to properly deploy and maintain the filter assembly 22 in place.

The filter cage assembly 24 may be configured with six self-expanding proximal struts 52, 53, 54, 55, 56, 58 and six self-expanding distal struts 62, 63, 64, 65, 66, 68. The particular size and shape of each strut can be varied (including, but not limited to, round, square and rectangular) without departing from the spirit and scope of the present invention. These proximal struts are part of the structure that forms the proximal portion 25 of the filter assembly 22, and the distal struts are part of the structure that forms the distal portion 23 of the filter assembly. Each proximal strut extends from one of six proximal apexes 142, 143, 144, 145, 146, 148 from the central open cell 40, and the distal struts extend from one of six distal apexes 242, 243, 244, 245, 246, 248 of the central open cell. The proximal struts have ends 152, 153, 154, 155, 156, 158 that may be configured for being retained at the proximal portion of the filter assembly, for example in the retaining collar 70 (FIGS. 1 and 2). The distal struts of the filter cage assembly may also be formed with distal ends 162, 163, 164, 165, 166, 168 that may are configured to be retained in the distal portion of the filter assembly, for example, in the obturator 32.

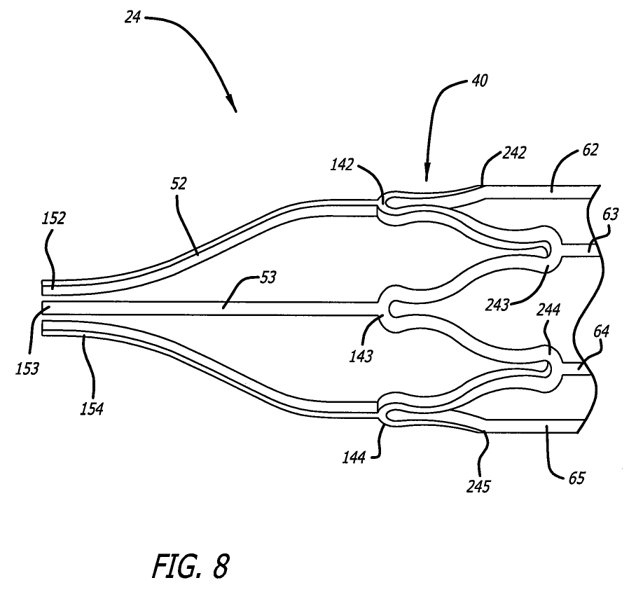

As shown in FIG. 8, the filter cage 24 may be laser cut from a tubular member and the proximal strut ends 152, 153, 154, 155, 156, 158 on the proximal struts 52, 53, 54, 55, 56, 58 may be mechanically bent to a smaller diameter than the diameter of the central open cell 40. The free ends of the proximal and distal struts are initially spaced apart after being formed from the tubular member (FIG. 7). The free ends of the proximal struts can be attached to a retaining collar or other device 70, such as is shown in FIGS. 1 and 2, to allow the expandable cage to be mounted to an elongated central member of the embolic filtering device 20, such as a guidewire 28. Alternatively, an inner sleeve and an outer sleeve may form a structure into which the proximal strut ends may be inserted and held in place around the guidewire. A known method of attaching the free ends of the proximal struts to the collar is with glue.

Alternatively, the proximal ends 152, 153, 154, 155, 156, 158 of the proximal struts 52, 53, 54, 55, 56, 58 may each have a partially cylindrical cross-section. This can be accomplished during the step of forming the filter cage assembly, wherein the filter cage is cut from a single cylinder of material. The proximal strut ends may be welded or otherwise joined together to form a proximal cylinder 34 (see FIGS. 5 and 6). This proximal cylinder acts as something similar to the retaining collar 70, such as is shown in FIGS. 1 and 2; however, the proximal cylinder is formed directly from the proximal strut ends. The strut ends may be welded or otherwise joined together along the seams formed along adjacent strut ends. In another approach, the proximal cylinder may be formed as a unitary structure as the filter cage is cut from a hypotube or otherwise formed from the substrate material.

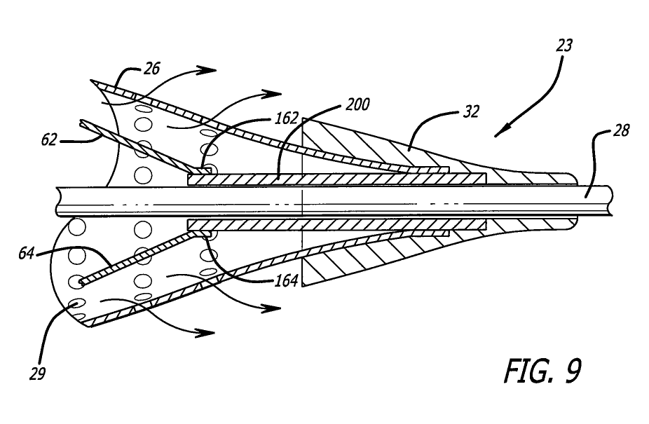

As shown in FIG. 9, the distal portion 23 of the embolic filter device 20 may be mounted on a guidewire 28. The filter sleeve 26, obturator 32 and the free ends 162, 163, 164, 165, 166, 168 of the distal struts 62, 63, 64, 65, 66, 68 may be secured to an inner collar or tubular member 200 with glue, by welding or by any other suitable process. Alternatively, each of the distal ends of the struts may be inserted in between an inner sleeve and an outer sleeve (not shown). The inner collar, together with the filter sleeve and the distal strut ends may slide longitudinally along the guidewire. Furthermore the inner collar may be configured to freely rotate about the guidewire, which assists the physician when the filter is deployed, particularly when the physician needs to twist the guidewire during the procedure.

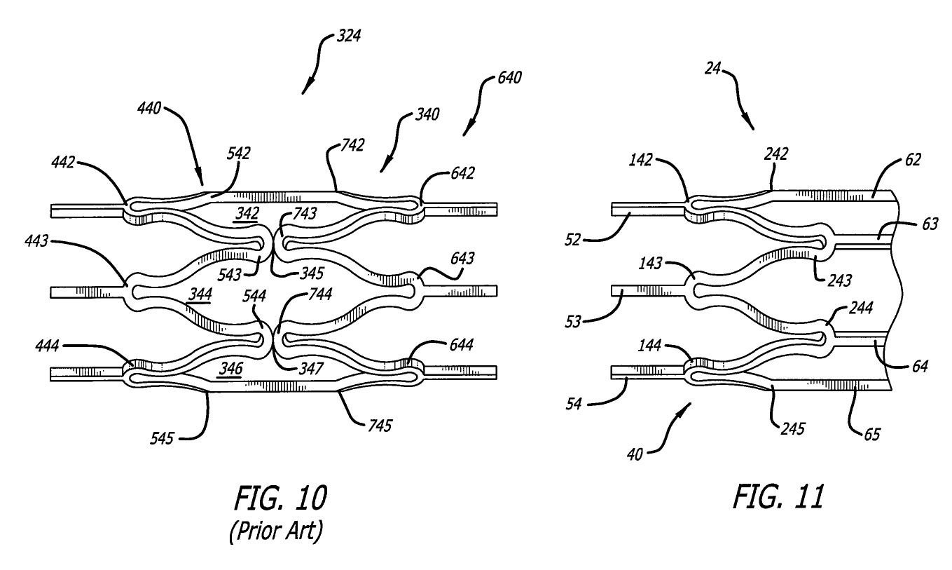

As shown in FIG. 10, a closed cell filter cage 324 is configured with a central closed cell ring 440; proximal vertices 440, 442, 443, 444, 742, 743, 744, 745 and distal vertices 542, 543, 544, 545, 642, 643, 644 constructed to form a closed loop in the shape of (but not limited to) a diamond, rhombus, kite, parallelogram or combinations thereof 342, 344, 346. A closed cell filter cage design can not be constructed from one ring. Closed cell designs are constructed from two sets of rings connected at every vertex 345, 347. In addition, a closed cell design must have at least two rings, forming looped geometry. Although a closed cell design may provide an excellent radial force, a closed cell design does not have enough flexibility so as to have a poor vasculature wall opposition due to the natural geometric properties of the closed cell. In addition, a closed cell configuration has a significant “landing zone length,” which also decreases flexibility and reduces vasculature wall opposition.

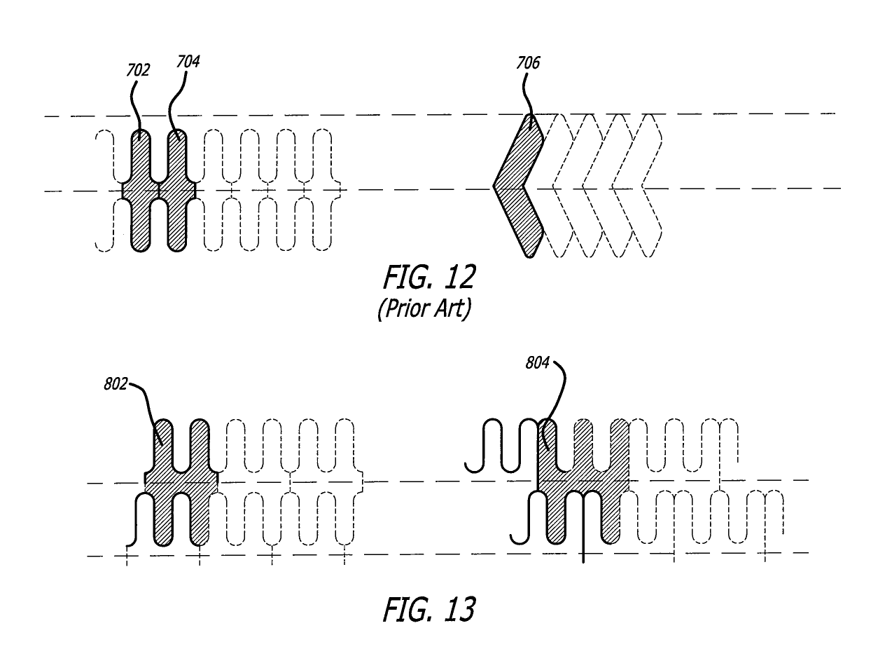

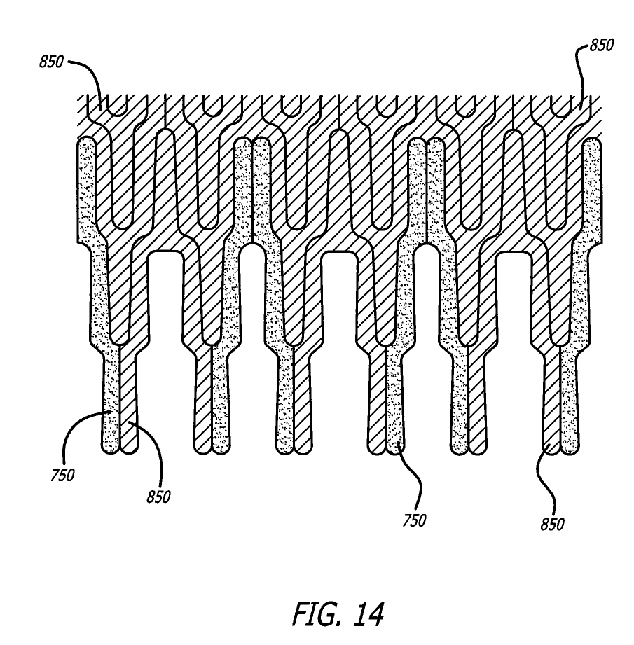

Conversely and referring to FIG. 11, an open cell filter cage 24 of the present invention is configured with a central open cell ring 40; proximal struts 52, 53, 54, 55, 56, 58 and distal struts 62, 63, 64, 65, 66, 68 constructed not to form a closed loop in the shape of (but not limited) the diamond, rhombus, kite, parallelogram etc. or their combinations. Such an open cell design may have at least one or more rings that preferably are not connected together at each vertex. An open cell filter cage design, however, can be constructed from one ring. Such an embolic filter cage will reduce the landing zone of the filter cage and will improve the contact of the filter cage to the vasculature wall. As shown in FIG. 12, closed cell designs 702, 704, 706 can not be constructed from one ring. A closed cell design constructed from two sets of rings is connected at every vertex. Closed cell designs reduced to one ring becomes an open cell design. Conversely and referring to FIG. 13, an open cell design 802, 804 can be constructed from one ring. Further examples of a closed cell configuration 750 and an open cell configuration 850 are shown in FIG. 14.

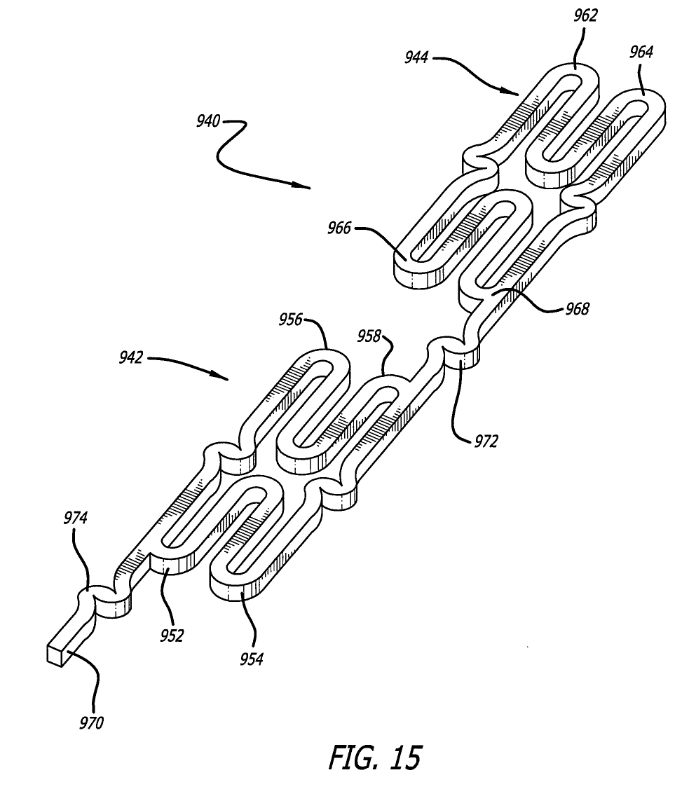

The filter cage assembly 940 of the present invention can be made in many ways. As shown in FIG. 15, one particular method is to cut a thin-walled tubular member, such as nickel-titanium, stainless steel or cobalt-chromium hypotube, to remove portions of the tubing in the desired pattern for each strut 968, 970. The laser cutting also forms the portions of the tubing for each apex 962, 964, 966, 954, 952; and may include two or more open cells 942, 944. The tubing may be cut into the desired pattern by means of a machine-controlled laser. Prior to laser cutting the strut pattern, the tubular member could be formed with varying wall thicknesses which will be used to create the flexing portions of the cage. The tubing used to make the cage could possibly be made of suitable biocompatible material such as spring steel.

The strut size is often very small, so the tubing from which the cage is made must necessarily have a small diameter. Typically, the tubing has an outer diameter of that of the final expanded cage and of the order of a few millimeters. Linear elastic tubing generally is lased full cage size tubing, and not expanded from a smaller size. Self-expanding stents of super elastic material are lased from small tubing and then mechanically set or heat set to a larger size. The wall thickness of the tubing is usually only a fraction of a millimeter. For cages implanted in body lumens, such as PTA applications, the dimensions of the tubing may be correspondingly larger.

While it is preferred that the cage be made from laser cut tubing, those skilled in the art will realize that the cage can be laser cut from a flat sheet and then rolled up in a cylindrical configuration with the longitudinal edges welded to form a cylindrical member. Also, the cage may be cut by other methods known in the art. If welded, the welding areas can be confined to non-bending areas of the cage so that the bending areas are outside the welded heat-effected zones.

Generally, the tubing is put in a rotatable collet fixture of a machine-controlled apparatus for positioning the tubing relative to a laser. According to machine-encoded instructions, the tubing is then rotated and moved longitudinally relative to the laser which is also machine-controlled. The laser selectively removes the material from the tubing by ablation and a pattern is cut into the tube. The tube is therefore cut into the discrete pattern of the finished struts. The cage can be laser cut much like a stent is laser cut. Details on how the tubing can be cut by a laser may be found in U.S. Pat. No. 6,131,266 (Saunders), which is hereby incorporated in its entirety by reference herein.

The process of cutting a pattern for the strut assembly into the tubing generally is automated, except for loading and unloading the length of tubing. For example, a pattern can be cut in tubing using a CNC-opposing collet fixture for axial rotation of the length of tubing, in conjunction with CNC X/Y table to move the length of tubing axially relative to a machine-controlled laser as described. The entire space between collets can be patterned using the CO₂ or Nd:YAG laser set-up. The program for control of the apparatus is dependent on the particular configuration used and the pattern to be ablated in the coding.

A suitable composition of nickel-titanium that can be used to manufacture the strut assembly of the present invention is approximately 55% nickel and 45% titanium (by weight) with trace amounts of other elements making up about 0.5% of the composition. The upper plateau strength is about a minimum of 60,000 psi with an ultimate tensile strength of a minimum of about 155,000 psi. The permanent set (after applying 8% strain and unloading), is approximately 0.5%. The breaking elongation is a minimum of 10%. It should be appreciated that other compositions of nickel-titanium can be utilized, as can other self-expanding alloys, to obtain the features of a self-expanding cage made in accordance with the present invention. That is, this is only one example of a suitable material, and other suitable material compositions known in the art or developed in the future may be used within the scope of the invention. For example, ELGILOY is another material that may be used to manufacture the filter cage. Also, very elastic polymers could be used to manufacture the filter cage.

The cage can also be manufactured by laser cutting a large diameter tubing of nickel-titanium which would create the cage in its expanded position. Thereafter, the formed cage could be placed in its unexpanded position by backloading the cage into a restraining sheath which will keep the device in the unexpanded position until it is ready for use.

In an alternative embodiment, the struts of the proximal strut assembly can be made from a different material than the distal strut assembly. In this manner, more or less flexibility for the proximal strut assembly can be obtained. When a different material is utilized for the struts of the distal proximal strut, the distal strut assembly can be manufactured through the process described above, with the struts of the proximal strut assembly being formed separately and attached to the distal assembly. Suitable fastening means such as adhesive bonding, brazing, soldering, welding and the like can be utilized in order to connect the proximal struts to the distal assembly. Suitable materials for the struts include materials such as nickel-titanium, spring steel, ELGILOY, along with polymeric materials which are sufficiently flexible and bendable.

Polymeric materials that can be utilized to create the filtering element include, but are not limited to, polyurethane, GORTEX, and ePTFE. The material can be elastic or non-elastic. The wall thickness of the filtering element is typically about 0.00050-0.0050 inches, although the wall thickness may vary depending on the particular material selected. The material can be made into a cone or similarly sized shape utilizing blow-mold or dip molding technology. The openings can be any different shape or size. A laser, a heated rod or other process can be utilized to create perfusion openings in the filter material. The holes, would of course, be properly sized to catch the particular size of embolic debris of interest.

The materials which can be utilized for the restraining sheath can be made from polymeric material such as cross-linked HDPE. This sheath can alternatively be made from a material such as polyolefin that has sufficient strength to hold the compressed strut assembly and has relatively low frictional characteristics to minimize any friction between the filtering assembly and the sheath. Friction can be further reduced by applying a coat of silicone lubricant, such as MICROGLIDE, to the inside surface of the restraining sheath before the sheaths are placed over the filtering assembly.

Regarding terminology, the term “tube” in the claims is not limited to circular cross-sections, but includes other closed cross-sections that may be useful in the context of this type of device, including square, triangular, or elliptical cross-sections and the like.

Further modifications and improvements may additionally be made to the device and method disclosed herein without departing from the scope of the present invention. Accordingly, it is not intended that the invention be limited, except as by the appended claims.

We claim:

1. An embolic filtering device, comprising:
a filter cage assembly that is movable between an unexpanded position and an expanded position, wherein the filter cage includes an open cell central ring, a plurality of proximal struts connected to the central ring and a plurality of distal struts connected to the central ring; and
a sleeve assembly formed around the distal struts of the filter cage assembly.

2. The embolic filtering device of claim 1, wherein the open cell central ring includes a plurality of distal vertexes attached to the distal struts.

3. The embolic filtering device of claim 2, wherein the sleeve assembly is secured to at least one of the distal vertexes of the open cell central ring.

4. The embolic filtering device of claim 1, wherein the open cell central ring includes a plurality of distal vertexes attached to each of the distal struts, and wherein the sleeve assembly is secured to each of the distal vertexes.

5. The embolic filtering device of claim 1, wherein the open cell central ring includes six proximal vertexes each joined to a proximal strut and six distal vertexes each joined to a distal strut, and wherein the sleeve assembly is secured to each of the distal vertexes.

6. The embolic filtering device of claim 5, wherein the sleeve assembly is configured with a plurality of perforations.

7. The embolic filtering device of claim 5, wherein the filter cage is formed from a nickel-titanium alloy.

8. The embolic filtering device of claim 5, wherein the filter cage is formed from stainless steel.

9. The embolic filtering device of claim 5, wherein the filter cage is formed from a cobalt-chromium alloy.

10. The embolic filtering device of claim 5, wherein the sleeve assembly is secured along a periphery of the open cell central ring.

11. The embolic filtering device of claim 1, wherein the sleeve assembly is secured along a periphery of the open cell central ring.

12. An embolic filtering device, comprising:
a filter cage assembly that is movable between an unexpanded position and an expanded position, wherein the filter cage includes an open cell central ring, a plurality of proximal struts each connected to a plurality of proximal vertexes formed in the central ring, and a plurality of distal struts connected to a plurality of distal vertexes formed in the central ring; and
a sleeve assembly formed around the distal struts of the filter cage assembly and secured to the open cell ring.

13. The embolic filtering device of claim 12, wherein the filter cage is formed with six proximal struts and with six distal struts, the open cell central ring is formed with six proximal vertexes and with six distal vertexes, and wherein the sleeve assembly is secured to each of the distal vertexes.

14. The embolic filtering device of claim 12, wherein the filter cage is formed with six proximal struts and with six distal struts, the open cell central ring is formed with six proximal vertexes and with six distal vertexes, and wherein the sleeve assembly is secured along a periphery of the open cell central ring.

15. A filtering system, comprising:
a filter cage assembly that is movable between an unexpanded position and an expanded position, wherein the filter cage includes an open cell central ring, a plurality of proximal struts each having a proximal portion and a distal portion such that each distal portion is connected to one of a plurality of proximal vertexes formed in the central ring, and a plurality of distal struts each having a proximal portion and a distal portion such that each proximal portion is connected to one of a plurality of distal vertexes formed in the central ring;
a sleeve assembly formed around the distal struts of the filter cage assembly and secured to the open cell ring;
a guidewire having a distal portion disposed within the filter cage and the sleeve assembly; and
a sheath configured to contain the filter cage and sleeve assembly when the filter cage is in the unexpanded position.

16. The filtering system of claim 15, wherein the sleeve assembly is secured to at least one of the distal vertexes.

17. The filtering system of claim 15, wherein the sleeve assembly is secured along a periphery of the open cell central ring.

18. The filtering system of claim 15, further including an obturator configured to enclose the distal portions of the distal struts.

19. The filtering system of claim 18, further including a cover configured to enclose the proximal portions of the proximal struts.

20. A method for positioning an embolic filter, comprising:
providing a filter cage assembly that is movable between an unexpanded position and an expanded position, wherein the filter cage includes an open cell central ring, a plurality of proximal struts each having a proximal portion and a distal portion such that each distal portion is connected to one of a plurality of proximal vertexes formed in the central ring, and a plurality of distal struts each having a proximal portion and a distal portion such that each proximal portion is connected to one of a plurality of distal vertexes formed in the central ring;
providing a sleeve assembly formed around the distal struts of the filter cage assembly and secured to the open cell ring;
providing a guidewire having a proximal portion and a distal portion disposed within the filter cage and the sleeve assembly;
providing a sheath configured to contain the filter cage and sleeve assembly when the filter cage is in the unexpanded position;
inserting the guidewire into the vasculature of a patient;
manipulating the proximal portion of the guidewire such that the distal portion of the guidewire is positioned at a desired location in the vasculature;
inserting the sheath containing the distal portion of the filter cage and sleeve assembly over the guidewire so as to position the distal portion of the sheath at the desired location in the vasculature; and
moving the sheath in a proximal direction so as to allow the filter cage to form in its expanded position.
